(19) United States
(12) Patent Application Publication
(10) Pub. No.: US 2010/0249232 A1
(43) Pub. Date: Sep. 30, 2010

(54) MOSQUITO REPELLENT

(75) Inventors: Hyouk II Kwoen, Seoul (KR); Kye Chung Park, Seoul (KR); Hyun Woo Oh, Daejeon (KR)

Correspondence Address: SHUTTS & BOWEN, LLP, 100 S. ASHLEY DRIVE, SUITE 1500, TAMPA, FL, 33602, US

(73) Assignee: BIO & HNT, Inc., Seoul (KR)

(21) Appl. No.: 12/600,346

(22) PCT Filed: May 31, 2007
(86) PCT No.: PCT/KR07/02659

§ 371 Date: Nov. 16, 2009

(30) Foreign Application Priority Data
May 17, 2007 (KR) 10-2007-0048299

(51) Int. Cl. A01N 37/06; C07C 57/03; A01P 7/04

(52) U.S. Cl. 514/560; 554/224

(57) ABSTRACT

The present invention relates to a mosquito repellent comprises at least one or more selected from the group consisting of neric acid and derivatives thereof in an effective amount to repel mosquito.

### TECHNICAL FIELD

The present invention relates to a mosquito repellent, and more particularly, to a new mosquito repellent composition comprising at least one compound selected from the group consisting of neric acid and derivatives thereof, which represent excellent mosquito repellent activity, as a mosquito repellent.

### BACKGROUND ART

Some insects are virulent and can cause harm, such as skin irritation, infection, and even disease, to people. In order to protect ourselves from such insects, various types of products have been developed. These products are developed to kill, suppress reproduction or proliferation, and debilitate sensory organs to maim perception.

Among these developed products, debilitation of sensory organs is used for a “repellent.” In particular, the repellent is often used for protecting ourselves from mosquitoes. Such a mosquito repellent can paralyze sensory organs of a mosquito and prevent the mosquito from tracing carbon dioxide or odor generated from the skin of a mammal, such as human beings.

Mosquito is a very harmful insect in view of hygiene since it can carry dangerous pathogen for causing disease such as dengue, yellow fever, encephalitis, and malaria. Considering that millions of people die worldwide every year from malaria, it is difficult to ignore the fact that mosquito is a deadly, harmful insect. It is reported that malaria has resurfaced and has been affecting people again since 1993, and malaria infected patients have recently increased to reach several thousand patients every year in Korea alone. Notably, the infection rate of malaria has increased every year and as can be inferred from the discussion above, direct or indirect contact with mosquitoes can have potentially dangerous consequence.

To combat mosquitoes, a mosquito repellent is preferably used because it is relatively free from harm to a human body. Various mosquito repellents are available. In particular, N,N-diethyl-m-toluamide (DEET) has been widely used owing to its excellent mosquito repellent effect. However, since the DEET has an unpleasant odor and strong penetration into the skin, it has been restricted from application on children, pregnant women, nursing mothers, hypotensive patients, people with sensitive skin, and so on. The United States restricts mosquito repellents containing more than 20% DEET from being manufactured.

In view of this, much efforts have been made to replace DEET. Although it is known that citronella, linalool, and lemongrass are effective in repelling mosquitoes, they are less effective than DEET. For this reason, a problem has been raised in practical use of mosquito repellents which contains the materials such as citronella, linalool, and lemongrass.

Accordingly, extensive studies and researches are made to develop new mosquito repellents derived from natural resources.

### DETAILED DESCRIPTION OF THE INVENTION

### Technical Problems

An objective of the present invention is to provide a mosquito repellent composition having excellent mosquito repellent effect.

Another objective of the present invention is to provide a mosquito repellent composition derived from natural resources being harmless to a human body.

Above and other objects of the present invention could be achieved by the present invention which will be described below.

### TECHNICAL SOLUTIONS

A mosquito repellent composition according to the present invention includes at least one mosquito repellent compound selected from the group consisting of neric acid and derivatives thereof.

The mosquito repellent compound is used more than 0.1%, and preferably in the range of 0.1 wt % to 25 wt % with respect to the total amount of composition, more preferably 1 wt % to 20 wt %.

The present invention includes a method of repelling mosquito, which comprises applying the mosquito repellent composition to the skin.

The mosquito repellent composition applied to the skin can be used in a type of a cream, a lotion, a spray, a spreader, or an ointment.

The present invention also includes a method of repelling mosquito, which comprises the steps of mixing a composition with a liquid solution, wherein the composition comprises at least one mosquito repellent compound selected from the group consisting of neric acid and derivatives thereof in an effective amount to repel mosquito, and applying the mixed liquid solution to a skin.

Hereinafter, the present invention will be described in detail.

### BEST MODE FOR CARRYING OUT THE INVENTION

In the present invention, it has been confirmed whether various natural resources that can be obtained from nature have mosquito repellent effect. As a result, it has been found that one or more chemical compounds selected from the group consisting of neric acid and derivatives thereof have repellency against mosquito.

The one or more compounds selected from the group consisting of neric and derivatives thereof are harmless to human body and have high repellent activity with respect to mosquitoes, especially Aedes.

Neric acid used in the present invention can be expressed as a molecular formula, i.e., C₁₀H₁₆O₂, and can have a following Chemical Structure 1.

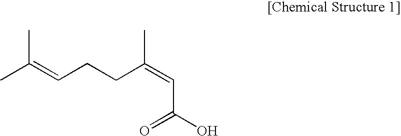

Another name of neric acid includes (Z,E)-3,7-dimethyl-2,6-octadienoic acid.

Neric acid is a known compound contained in plant essential oils such as lemon mint whose botanical nomenclature is Satureja punctata [Lameck S. Chagonda and Jean-Claude Chalchat, The composition of the leaf essential oil of Satureja punctata (Benth.) Brig. From Zimbabwe, Flavour and Fragrance Journal, Vol. 20, 2005, pp. 316-317] and sweet orange whose botanical nomenclature is Citrus sinensis [Pin Yang, Yajun Ma and Shuiqing Zheng, Adulticidal Activity of Five Essential Oils against Culex pipiens quinquefasciatus, Journal of Pesticide Science, Vol. 30, 2005, pp. 84-89]. Neric acid is commercially available.

The chemical structures of the aforementioned neric acid can be modified to manufacture derivatives for a new repellent which represents high repellent activity to mosquitoes. In the present invention, the derivatives are compounds derived from neric acid as an initiative reactant by the method like substituting one or more elements of neric acid and so on.

In the present invention, compounds used as the aforementioned mosquito repellent are used alone or mixed with a well-known solvent to manufacture a mosquito repellent composition. The well-known solvent means either a solvent used for a general repellent composition or a doping material applied to the skin of a human body, having a type of a cream, a liquid phase, a spray, and a gel. In this case, the aforementioned neric acid and derivatives thereof are used alone or in combination. In this case, the aforementioned neric acid and derivatives thereof are used more than 0.1 wt % with respect to a weight of the total mosquito repellent composition, preferably in the range of 0.1 wt % to 25 wt %, more preferably in the range of 1 wt % to 20 wt %.

The mosquito repellent composition according to the present invention is preferably used in such a manner that it is applied to the skin in a type of a cream, a lotion, a spray, a spreader, or an ointment. The mosquito repellent composition is used in a place where mosquitoes inhabit. The expression, “applied to the skin” includes “applied to a path of a mosquito for contact to the skin, such as clothes and doors,” as well as “directly applied to the skin.” Furthermore, it is expected that the mosquito repellent composition can be used as a repellent for arthropod of various species in addition to mosquito.

The present invention will be embodied by the following embodiment, and the following embodiment is only a detailed exemplary of the present invention and is not intended to restrict or limit the protection scope of the present invention.

### MODE FOR CARRYING OUT THE INVENTION

### Embodiment

### Preparation of Sample

Neric acid which is used in the present invention, are commercially available or can directly be synthesized. In this embodiment, products of Bedoukian Research, Inc. (product name is Neric Acid) were purchased and used as neric acid.

Purities of the above three compounds were examined using a gas chromatograph (GC) and a gas chromatograph-mass spectrometry (GC-MS). The compounds having purity of 99.5% or greater were only used. Respective ingredients of the above compounds were added to isopropyl alcohol of 91% to make test samples consisting of a solution at the concentration of 2% and a solution at the concentration of 10%. The samples were used for mosquito repellent efficacy experiments.

The three test samples manufactured as above underwent a repellent effective experiment with respect to Aedes which is a test insect for examining mosquito repellency.

Examination Test Mosquitoes Used in the Experiments

In this embodiment, Aedes aegypti was used test mosquitoes. The Aedes aegypti was supplied from Korea Research Institute of Bioscience and Biotechnology and reared indoor from generation to generation.

The larvae of the Aedes aeqypti were reared in a plastic container of 22×14×7 cm containing distilled water with a one to one mixture of chicken feed and yeast. Mosquito pupae were transferred into a paper cup and then placed in a rearing cage to emerge as adults. Here, the rearing cage was constructed with metal net. Males and females of the adult mosquitoes at one to one ratio were put in a rearing cage for two or three days for mating. The female mosquitoes were then used for repellency tests. Eight percent (8%) sugar water was supplied for the adult mosquitoes. The following rearing conditions were maintained: light condition of 16 h:8 h (day:night), temperature of 27±2° C., and relative humidity of 80±10%.

Examination of Repellent Activity Using Skin Test

In order to examine effectiveness of the mosquito repellents of the prepared sample solutions, one (1) ml of a sample solution was uniformly applied, using a metal rod, on a forearm of a volunteer human test subject between a wrist and an elbow. Thereafter, the arm treated with the test solution was inserted in a bioassay cage constructed with Plexiglass of 50×50×50 cm containing approximately 100 mated female mosquitoes. The number of mosquitoes trying to bite was recorded for three minutes in every thirty minutes for up to 4 hours. The time required to get the arm bitten by two cumulative mosquitoes was regarded as effective repellency duration. A net window of 40×40 cm was present at each side and top of the Plexiglass bioassay cage to allow air ventilation. A 15×15 cm hole was prepared at the front side of the Plexiglass bioassay cage and a long net sleeve was attached to the hole so that the test arm could be put into the cage for repellency test.

At the beginning of each repellency test, an arm treated with solvent only was placed in the bioassay cage containing mosquitoes, in order to examine blood feeding activity of the mated female test mosquitoes. This test with the bioassay cage was carried out only when the arm was bitten by at least two mosquitoes for initial three minutes.

Result of Examination of Repellent Activity of Each Ingredient

Repellent activity against Aedes was examined in accordance with the aforementioned method using the samples which respectively contain neric acid. Also, mosquito repellent activity was measured with solutions which contain DEET and Picadirin, wherein the DEET and Picadirin are commercially available mosquito repellents which are currently recommended by the Center for Disease Control of the United States. The measured results were shown in Table 1 to Table 4.

TABLE 1

| | | Effective repellency duration (minute) |
|---|---|---|
| Compound | Concentration | (average ± standard deviation) |
| Neric acid | 2% | 106 ± 16.3 |
| DEET | 2% | 70 ± 10.0 |

Table 1 indicates effective repellency duration of each compound at the concentration of 2%, represented as protection time until the arm was bitten by two cumulative mosquitoes. In this case, the dose amount of the concentration of 2% corresponds to 0.05 mg/cm³.

TABLE 2

| | | Effective repellency duration (minute) |
|---|---|---|
| Compound | Concentration | (average ± standard deviation) |
| Neric acid | 10% | 240 ± 0 |
| DEET | 10% | 218 ± 10.8 |
| Picadirin | 10% | 158 ± 27.5 |

Table 2 indicates effective repellency duration of each compound at the concentration of 10%, represented as protection time until the arm was bitten by two cumulative mosquitoes. In this case, the dose amount of the concentration of 10% corresponds to 0.25 mg/cm³, and the maximum measurement time was 240 minutes.

As shown in Table 2 above, effective repellency duration of neric acid lasted for 240 minutes at the level of 0.25 mg/cm³. It is noted that neric acid have greater mosquito repellent effect than that of DEET or Picadirin whereas effective repellency duration of DEET lasted for 218 minutes and the effective repellency duration of Picadirin lasted for 158 minutes at the same level.

As shown also in Table 1 above, effective repellency duration of neric acid lasted for 106 minutes at the level of 0.05 mg/cm³ whereas effective repellency duration of DEET lasted for 70 minutes at the same level.

TABLE 3

| | | Repellent efficiency (%) per time | | | | | | | |
|---|---|---|---|---|---|---|---|---|---|
| | | 30 | 60 | 90 | 120 | 150 | 180 | 210 | 240 |
| Compound | Concentration | minutes | minutes | minutes | minutes | minutes | minutes | minutes | minutes |
| Neric acid | 2% | 100 | 83.1 | 70.4 | 52.1 | 39.4 | 26.8 | 18.3 | 14.1 |
| DEET | 2% | 100 | 0 | 0 | 0 | 0 | 0 | 0 | 0 |

Table 3 indicates a repellent efficiency measured at every 30 minutes after test solution was applied onto an arm at the concentration of 2%. In this case, the dose amount of the concentration of 2% corresponds to 0.05 mg/cm³.

TABLE 4

| | | Repellent efficiency (%) per time | | | | | | | |
|---|---|---|---|---|---|---|---|---|---|
| | | 30 | 60 | 90 | 120 | 150 | 180 | 210 | 240 |
| Compound | Concentration | minutes | minutes | minutes | minutes | minutes | minutes | minutes | minutes |
| Neric acid | 10% | 100 | 100 | 100 | 100 | 100 | 100 | 100 | 95.3 |
| DEET | 10% | 100 | 100 | 100 | 100 | 87.5 | 81.3 | 78.1 | 71.9 |
| Picadirin | 10% | 85.0 | 85.0 | 85.0 | 80.0 | 75.0 | 72.5 | 62.5 | 47.5 |

Table 4 indicates a repellent efficiency measured at every 30 minutes after test solution was applied onto an arm at the concentration of 10%. In this case, the dose amount of the concentration of 10% corresponds to 0.25 mg/cm³.

In Table 3 and Table 4, the repellent efficiency was calculated using the following Equation 1.

$$Repellent efficiency ( \% ) = \frac{( \begin{matrix}number of mosquitoes of control example - \\ number of mosquitoes of \\ experiment example\end{matrix} )}{number of mosquitoes of control example} \qquad [ Equation 1 ]$$

Based on the results of Table 3 and Table 4 above, neric acid indicated strong repellent efficiency of 100% for 30 minutes at the level of 0.05 mg/cm³. Moreover, the results indicated high repellent effect greater than that of DEET even after lapse of 30 minutes. Moreover, based on the results of Table 3 and Table 4 above, neric acid indicated strong repellent efficiency of 100% for 210 minutes at the level of 0.25 mg/cm³. As a result, it is noted that neric acid indicated greater repellent activity than that of DEET or Picadirin.

It will be apparent to those skilled in the art that the present invention can be embodied in other specific forms without departing from the spirit and essential characteristics of the invention.

1. A mosquito repellent composition comprising at least one mosquito repellent compound selected from the group consisting of neric acid and derivatives thereof in an effective amount to repel mosquito.

2. The composition as defined in claim 1, wherein the neric acid has a following Chemical Structure 1:

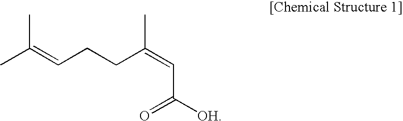

3. The composition as defined in claim 1, wherein the mosquito repellent compound is used in the range of 0.1 wt % to 25 wt % with respect to the total amount of composition.

4. The composition as defined in claim 2, wherein the mosquito repellent compound is used in the range of 0.1 wt % to 25 wt % with respect to the total amount of composition.

5. The composition as defined in claim 3, wherein the mosquito repellent compound is used in the range of 1 wt % to 20 wt % with respect to the total amount of composition.

6. The composition as defined in claim 4, wherein the mosquito repellent compound is used in the range of 1 wt % to 20 wt % with respect to the total amount of composition.

7. A method of repelling mosquito, which comprises applying mosquito repellent composition comprising at least one mosquito repellent compound selected from the group consisting of neric acid and derivatives thereof in an effective amount to repel mosquito.

8. A method of repelling mosquito, which comprises applying mosquito repellent composition according to claim 7, wherein the neric acid has a following Chemical Structure 1:

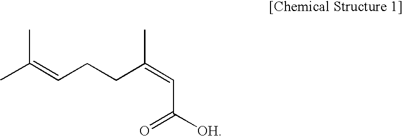

9. A method of repelling mosquito, which comprises applying mosquito repellent composition according to claim 7, wherein the mosquito repellent compound is used in the range of 0.1 wt % to 25 wt % with respect to the total amount of composition.

10. A method of repelling mosquito, which comprises applying mosquito repellent composition according to claim 8, wherein the mosquito repellent compound is used in the range of 0.1 wt % to 25 wt % with respect to the total amount of composition.

11. A method of repelling mosquito, which comprises applying mosquito repellent composition according to claim 9, wherein the mosquito repellent compound is used in the range of 1 wt % to 20 wt % with respect to the total amount of composition.

12. A method of repelling mosquito, which comprises applying mosquito repellent composition according to claim 10, wherein the mosquito repellent compound is used in the range of 1 wt % to 20 wt % with respect to the total amount of composition.

13. The method as defined in claim 7, wherein the composition is applied to the skin in a type of a cream, a lotion, a spray, a spreader, or an ointment.

14. The method as defined in claim 8, wherein the composition is applied to the skin in a type of a cream, a lotion, a spray, a spreader, or an ointment.

15. The method as defined in claim 9, wherein the composition is applied to the skin in a type of a cream, a lotion, a spray, a spreader, or an ointment.

16. The method as defined in claim 10, wherein the composition is applied to the skin in a type of a cream, a lotion, a spray, a spreader, or an ointment.

17. The method as defined in claim 11, wherein the composition is applied to the skin in a type of a cream, a lotion, a spray, a spreader, or an ointment.

18. The method as defined in claim 12, wherein the composition is applied to the skin in a type of a cream, a lotion, a spray, a spreader, or an ointment.

19. A method of repelling mosquito, which comprises the steps of:
mixing a composition with a liquid solution, wherein the composition comprises at least one mosquito repellent compound selected from the group consisting of neric acid and derivatives thereof in an effective amount to repel mosquito; and
applying the mixed liquid solution to a skin.

20. The method as defined in claim 19, wherein the neric acid has a following Chemical Structure 1:

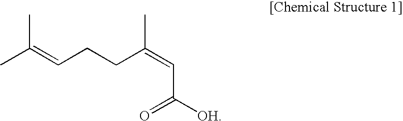

21. The method as defined in claim 19, wherein the mosquito repellent compound is used in the range of 0.1 wt % to 25 wt % with respect to the total amount of composition.

22. The method as defined in claim 20, wherein the mosquito repellent compound is used in the range of 0.1 wt % to 25 wt % with respect to the total amount of composition.

23. The method as defined in claim 21, wherein the mosquito repellent compound is used in the range of 1 wt % to 20 wt % with respect to the total amount of composition.

24. The method as defined in claim 22, wherein the mosquito repellent compound is used in the range of 1 wt % to 20 wt % with respect to the total amount of composition.

25. The method as defined in claim 19, wherein the composition is applied to the skin in a type of a cream, a lotion, a spray, a spreader, or an ointment.

26. The method as defined in claim 20, wherein the composition is applied to the skin in a type of a cream, a lotion, a spray, a spreader, or an ointment.

27. The method as defined in claim 21, wherein the composition is applied to the skin in a type of a cream, a lotion, a spray, a spreader, or an ointment.

28. The method as defined in claim 22, wherein the composition is applied to the skin in a type of a cream, a lotion, a spray, a spreader, or an ointment.

29. The method as defined in claim 23, wherein the composition is applied to the skin in a type of a cream, a lotion, a spray, a spreader, or an ointment.

30. The method as defined in claim 24, wherein the composition is applied to the skin in a type of a cream, a lotion, a spray, a spreader, or an ointment.

31. A use of at least one compound selected from the group consisting of neric acid and derivatives thereof as a mosquito repellent.
